(19) United States
(12) Patent Application Publication
(10) Pub. No.: US 2025/0250549 A1
(43) Pub. Date: Aug. 7, 2025

(54) SYSTEMS AND METHODS FOR VIRAL TRANSFECTION OF CELLS IN A BIOREACTOR

(71) Applicant: Culture Biosciences, Inc., South San Francisco, CA (US)

(72) Inventors: William Patrick, South San Francisco, CA (US); Babu Sivaraman, South San Francisco, CA (US); Jeff Thayer, South San Francisco, CA (US); Helen Luo, South San Francisco, CA (US); Collin Edington, South San Francisco, CA (US); Nicholas Lester, South San Francisco, CA (US)

(73) Assignee: Culture Biosciences, Inc., South San Francisco, CA (US)

(21) Appl. No.: 19/041,898

(22) Filed: Jan. 30, 2025

Related U.S. Application Data
(60) Provisional application No. 63/549,387, filed on Feb. 2, 2024.

(51) Int. Cl. C12N 7/00; C12M 1/00; C12M 1/34; C12N 15/85; C12N 15/86

(52) U.S. Cl. CPC C12N 7/00; C12M 29/06; C12M 41/00; C12N 15/85; C12N 15/86; C12N 2510/02; C12N 2710/10043; C12N 2710/10052; C12N 2710/14043; C12N 2710/14052; C12N 2710/16643; C12N 2710/16652; C12N 2740/10043; C12N 2740/10052; C12N 2740/15043; C12N 2740/15052; C12N 2750/14143; C12N 2750/14152; C12N 2800/107

(57) ABSTRACT

Embodiments provide systems and methods for transfecting cells to produce a viral vector or other selected virus. One embodiment of a method for transfecting cells to produce a selected virus includes providing a first solution comprising plasmids or other extrachromosomal DNA encoding the virus and a second solution comprising a transfection agent; the two solutions kept separate. The two solutions are then mixed to produce a transfection solution (TS) to be delivered to a cell culture medium (CCM) for transfection of cells in the medium with the plasmid DNA encoding the virus wherein the mixing is initiated responsive to a trigger event. The TS is then incubated to form transfection complexes. Subsequently, the TS is delivered to the CCM to transfect the cells in the medium wherein a viral production parameter resulting from transfection is optimized by initiating mixing of the solutions responsive to the trigger event.

### CROSS-REFERENCE TO RELATED APPLICATIONS

This application claims the benefit of and priority to U.S. Provisional Patent Application Ser. No. 63/549,387 filed on Feb. 2, 2024 and titled “Systems And Methods For Viral Transfection Of Cells In A Bioreactor, the contents of which are incorporated herein in their entirely for all purposes. This application is also related to U.S. Provisional Patent Application Ser. No. 63/552,997, filed on Feb. 13, 2024 and titled Apparatus, System, And Method For Automated Filling Of Syringes the contents of which are incorporated herein for all purposes.

### BACKGROUND

Viral transfection is a process where viruses are used as tools to introduce genetic material into cells. The method is widely used for research and therapeutic applications including gene therapy and DNA vaccination. The first step in the process is the production of viral vectors which are viruses that have been genetically modified to carry foreign genetic material in the form of one or more transgenes which is/are then inserted into a host cell upon infection of the host cell to modify the cell in the desired manner. Viral vectors are typically produced by transfection of mammalian cells known as packaging cells with plasmids or other extrachromosomal DNA molecules encoding the virus. This process involves adding the plasmids along with agents known as transfection agents to facilitate transfection of the cells with the plasmid to a cell culture medium containing the packaging cells at selected stages in the growth of the cell culture during the cell culture process. Upon taking up the plasmid DNA, the cellular mechanisms of the packaging cells produce and release multiple copies of the virus typically upon death of the cell which are then harvested and purified using methods known in the art.

Advances in the use of engineered viral vectors have created a need for large-scale manufacture of clinical or other high grade viral vectors particularly for gene therapy, DNA vaccination and other clinical and research applications. Viral vectors employing adeno-associated viruses, lenti viruses, onco-retroviruses and other viruses have gained momentum as an effective means for delivering genetic material into cells and are used in cell and gene therapies as well as vaccines production. Currently, scalable generation of viral vectors for delivering genetic information (e.g., for gene therapy, vaccines, cancer therapy, research and other applications) is typically performed in suspension-based mammalian cell cultures using stirred tank reactors. HEK293 and derivative cell lines are the predominant cell lines used as the viral production platform and transient transfection of HEK293 cells within the bioreactor remains the most efficient way to generate the viral particles also known as virions.

However, there are a number of issues with current transfection methods for the production of viral vectors. First the viral vector production process is vastly more complex than traditional recombinant protein production using mammalian cell cultures in a stirred tank and requires a specialized labor-intensive workflow for the transient transfection process workflow. Also, HEK293 platforms for viral vector production are still not as well characterized as their protein expression CHO cell line counterparts resulting in inconsistent results for the production of just one viral vector for one specific therapy. The problem is compounded when the same or different vectors are produced for different therapies or other applications. This is due to the fact that despite years of use, there is no one-size fits all process for viral vector development, as factors unique to each therapy or vaccine impact the optimal process parameters, and therefore the critical quality attributes of the final product, such as overall yield and full/empty capsid ratio. Factors include cell line, culture conditions, vector design, payload size, promoters and enhancers, transfection efficiency, time of harvest, and more.

In addition to the above issues, current transfection methods yield inconsistent results and/or low transfection efficiency because they involve a manual approach for mixing solutions containing plasmids and the transfection agents and adding them to the packaging cell culture. There are at least three reasons for this. First, inadequate or inconsistent mixing of the plasmids and transfection agent solution can reduce the effectiveness of transfection of the packaging cells. Second, the mixed solution begins to degrade after time so if it is not added in sufficient time, transfection effectiveness will be reduced. Third, it is desirable to be able to add the mixed solutions of plasmids and transfection agents to the culture of packaging cells at a reproducible point in the life of the packaging cell culture. Doing so however, requires taking one or more measurements of the packing cell culture solution, then potentially doing analysis to determine the desired time for adding the mixed transfection solution all of which can introduce significant variation in the point at which the mixed solution is added. Thus, manually mixing the plasmid and transfection agent solutions and then adding the mixed solution to the packaging cell culture can introduce significant amounts of batch-to-batch variation in the transfection process and resulting production of the viral vectors including in one or more of yield, purity, genetic composition and infectivity of the resulting viral vectors. Variation in one or more of these factors can in turn adversely affect the in vivo efficaciousness of the vector (e.g., its ability to both infect and transmit its genetic payload material to the host cell) and/or cause other adverse in vivo effects including immunogenicity and/or cytotoxicity when the vector infects the target in vivo cells. Thus, there is a need for more improved methods for transfection of the production of viral vectors for gene therapy and other applications.

### BRIEF DESCRIPTION OF THE DRAWINGS

Embodiments of the subject matter disclosed herein in accordance with the present disclosure will be described with reference to the drawings, in which:

FIG. 1 is a schematic illustrating a system for automated transfection of a cell culture with DNA encoding a viral vector according to an embodiment of the subject matter disclosed herein.

FIG. 2 is a schematic illustrating a system for automated transfection of a cell culture with DNA encoding a viral vector using syringe pumps for the delivery of one or more solutions used for transfection of a cell culture according to an embodiment of the subject matter disclosed herein.

FIG. 3 is a block diagram illustrating a cloud based system for automated transfection of a cell culture to produce viral vectors the system including software and firmware feedback control systems according to an embodiment of the subject matter disclosed herein.

FIG. 4 is a schematic illustrating a system for automated transfection of a cell culture wherein the system includes a mixing device according to an embodiment of the subject matter disclosed herein.

FIGS. 5a-b are lateral views illustrating embodiments of a mixing device, FIG. 5a illustrates a mixing device, FIG. 5b illustrates a mixing device with an internal mixing chamber having a helical pattern according to an embodiment of the subject matter disclosed herein.

FIGS. 6a-6b are schematics illustrating preparation of plasmid (FIG. 6a) and transfection agent (FIG. 6b) solutions using an auto-filling device according to an embodiment of the subject matter disclosed herein.

FIG. 7 is a schematic illustrating a transfection system using a microfluidic chip to prepare of one or more solutions used for transfection of cell culture with DNA encoding a viral vector wherein the microfluidic chip is fluidically coupled to syringes for storing, mixing and dispensing of the solutions used for the transfection process according to an embodiment of the subject matter disclosed herein.

FIG. 8 is a flow chart illustrating a method of automating one or more aspects of a process for transfecting a cell culture to produce a viral vector according to an embodiment of the subject matter disclosed herein

FIG. 9 is a flow chart illustrating an embodiment of a method of using embodiments of systems for automating one or more aspects of a process for transfecting a cell culture to produce a viral vector according to an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

By way of an overview, embodiments described herein relate to systems and methods for transfection of cells with genetic delivery agents containing foreign genetic material. More specifically, embodiments relate to the systems and methods for the transfection of cells by genetic delivery agents encoding DNA for selected viruses. embodiments provide systems and methods for the transfection of mammalian and other eukaryotic cells for the production of viral vectors for use in various research and medical applications including gene therapy and DNA vaccination. Many embodiments provide methods for such viral transfection where one or more aspects of the process for example mixing and incubation of the transfection solution are controlled and/or automated using a controller such as a microprocessor or other logic resources.

In a first aspect, embodiments provide methods for the transfection of mammalian or other cells to produce a selected virus which, in many embodiments, may correspond to a viral vector configured to deliver genetic coding material to target cells within a human or other mammal or organism. One embodiment of such a method comprises providing a first solution comprising a plasmid or other extrachromosomal DNA encoding a virus and a second solution comprising a transfection agent wherein the first and second solutions are kept separate. Then two solutions are mixed to produce a transfection solution to be delivered to a cell culture medium for transfection of cells in the medium with the extrachromosomal DNA wherein the mixing is initiated responsive to a trigger event. The transfection solution is then incubated to form transfection complexes which facilitate uptake of the extrachromosomal DNA into cells. Subsequently, the transfection solution is delivered to the cell culture medium to transfect the cells in the medium wherein a viral production parameter resulting from transfection of the cells is optimized by initiating the mixing of the first and second solutions responsive to the trigger event.

According to one or more embodiments, the viral production parameter may correspond to one or more of a viral concentration, a batch-to-batch variability of viral concentration, an amount of variation in the genetic composition of a population of viral particles from a production batch, an average virus infectivity of a production batch or ratio of full to empty capsids for a production batch For embodiments utilizing viral concentration or derivative property as the viral production parameter, the concentration may correspond to physical titer or functional titer, the former measured in gc or vg/ml and the latter in transductions unit (TU) per ml, plaque-forming units per mL (pfu/mL), or infectious units per mL (ifu/mL), depending on the viral vector.

Typically, the virus will comprise a genetically modified virus which is engineered to deliver one or more genes or other genetic material to target cells in a human, mammal or other animal or organism upon infection of the target cells with the virus. Such viruses are also referred herein as viral vectors. In many embodiments, the one or more genes are selected/engineered to treat a genetic disease or condition such as sickle cell anemia, hemophilia and or various glycogen storage disorders such as Pompe Disease. In various embodiments, the genetically modified virus may correspond to one or more of an adenovirus, an adeno-associated virus, a lentivirus, a retrovirus, an onco-retrovirus or other viruses known in the gene therapy and viral transfection arts.

In many embodiments, the extrachromosomal DNA molecules correspond to plasmids, however other forms of extrachromosomal DNA known in the art may also be used. In some embodiments, all the plasmids in solution may essentially be identical and carry the same DNA encoding the same genes while in others the first solution also described herein as the plasmid solution may include different plasmids each including DNA for a different gene(s) or viral genomes. In specific embodiments, the plasmid solution may comprise between 2 to 20 different plasmids having different DNA encoding different genes and/or viral genomes.

In various embodiments the transfection agent may comprise lipid and/or liposomes selected to increase the efficiency of transfection of the selected mammalian or other eukaryotic cells with the plasmid or other extrachromosomal DNA. In particular embodiments, the transfection agent comprises lipofectamine which contains cationic liposomes which form complexes with the plasmid (or other extrachromosomal DNA wherein the plasmids are entrapped within the liposome. The liposome plasmid complexes (also referred to herein as transfection complexes) then fuse and cross the wall of the cell to be transfected allowing the plasmid DNA cargo to get into the cell cytoplasm for expression within the cell. The use of other liposomes including cationic liposomes known in the art as transfection agents is also contemplated. In use, these or other transfection agents serve to increase the efficiency of transfection of the target cells with the selected plasmid or other extrachromosomal DNA. Also in various embodiments, one or more of the transfection agent and its concentration within the transfection solution can be selected and/or titrated to achieve a desired transfection efficiency depending on one more of the plasmid DNA encoding the virus, the packaging cell line and the packaging cell culture conditions.

In other embodiments, the transfection agent may comprise various polymers which bind negatively charged DNA and impart a cationic charge to the DNA so that it can transit through cell membranes and enter the cell cytoplasm to the cell nucleus. An exemplary embodiment of such a polymer is polyethylenimine (PEI) transfection. PEI is a synthetic polymer with an exceptionally high positive charge density in pH-neutral solutions. Positively charged PEI binds strongly to negatively charged DNA and imparts a net cationic charge, allowing the DNA to enter cells. PEI can be used as the transfection agent in a number of embodiments particularly those employing HEK293 as the packaging cell line to be transfected,

The trigger event (also referred to herein as a trigger point) may correspond to a variety of events related to the cell culture medium and/or the process for preparing and incubating the cell culture medium with packaging cells used for production of the selected virus. For example, in some embodiments, the trigger event is a time period associated with the cell culture medium such as the amount of time after inoculation of the cell culture with the packaging cells (i.e., the amount of time after an inoculum of packaging cells are added to the cell culture medium).

In other embodiments, the trigger event corresponds to a biological condition(s) of the cell culture medium and/or an amount or rate(s) of change of the biological condition(s). In particular embodiments, the trigger event corresponds to a density of packaging cells within the cell culture medium. Typically, in such embodiments, the trigger event is the reaching of a threshold upper limit of cell density and/or the reaching and maintaining of cell density above a lower limit and below an upper limit for a select period of time. In related embodiments, it may also correspond to an amount of change of cell density and/or a rate of change of cell density which, in either case, may be a minimum or maximum change or rate of change. Typically, the amount or rate of change will be a positive value reflecting a state of cell growth within the culture medium; however, in some embodiments, it may correspond to a negative value indicating, for example, that the cell culture had reached a plateau in growth and is now starting to decrease in cell density. The particular positive or negative value in the amount or rate of change of cell density used as the trigger point in a particular batch run can be selected based on one or more factors including the following: selected viral vector, packaging cell line, cell culture medium and volume of the bioreactor vessel. Cell density can be measured using various means including optical means. In particular embodiments, cell density is measured using optical turbidity techniques known in the art. Also in various embodiments, such turbidity or other cell density measurements may be made continuously or at selected intervals under the control of a processor or other controller described herein.

In other embodiments, the biological condition used for the trigger event may include one or more of pH, oxygen concentration, CO2 concentration or glucose concentration of the cell culture solution. Such measurements may also be selectively grouped together to create a composite or multifactor measurement of the cell culture medium providing information on an overall and/or multifactor state of cell culture where optimal transfection can occur.

In use, such embodiments using cell density and/or related properties as the trigger event allow the transfection solution including the transfection complexes to be added to the cell culture medium in a time window where optimal transfection efficiency of the packaging cells (with the plasmid/extrachromosomal DNA encoding the virus) can be achieved. (Transfection efficiency being defined in some embodiments as the number of packaging cells expressing the plasmid DNA encoding the virus divided by the total number of packaging cells in the cell culture medium.). In various embodiments, the triggering cell density can range from about one million to two million cells per ml with large and smaller ranges also considered depending on various factors. For example, in some embodiments, the triggering cell density can range from about 0.5 to 5, 1 to 4, 1 to 3, 2 to 5, or 2 to 4 million viable cells per ml. In particular embodiments where the bioreactor and cell culture process is operated in a perfusion mode (e.g., where nutrient solutions are added and waste products removed in a continuous and/or frequent basis) the triggering cell density can be in the range of 3 to 10 million, 4 to 10 million and 6 to 10 million viable cells per ml with particular embodiments of 4, 6 and 8 million viable cells per ml. In various embodiments the triggering cell density can be selected based on one or more of the following factors: the cell line used for the packaging cells, the cell culture medium, the specific virus to be produced by the packaging cells, the desired viral concentration (e.g., one or both of physical and functional titer) or the total amount of viral particles to be produced (e.g., 109, 10 10, and the like), the size of the bioreactor vessel (250 ml vs 5 L or 10 L or larger) or mixing speed within the bioreactor.

In various embodiments, the packaging cell line used to generate the selected virus corresponds to various mammalian cell lines known in the cell culture arts including one or more of embryonic cells, renal cells or embryonic renal cells one or more of which may be immortal cell lines. In particular embodiments, the mammalian cells used to generate the selected virus comprise HEK 293 cells known in the cell culture arts. In still other embodiment, the cells used for the packaging cells may comprise other eukaryotic cells besides mammalian cells including yeast cells.

In another aspect, embodiments provide systems for transfecting cells to produce a selected virus wherein the system is configured to automate and/or control one or more aspects of the process. One embodiment of such a system comprises a first fluid delivery device configured to contain and deliver a first solution comprising extrachromosomal DNA molecules e.g., plasmids encoding the virus and a second fluid delivery device configured to contain and deliver a second solution comprising a transfection agent. A first fluid delivery channel is fluidically coupled to the first and second fluid delivery devices and a control valve is fluidically coupled to at least one of the first and second fluid delivery devices and the first fluidic delivery channel. The control valve is configured to control flow between the first and second fluid delivery devices and outward flow from one or both of the fluid delivery devices to a second or another fluid delivery channel. In many embodiments, the control valve is operably coupled to a controller which is configured to control the valve so as control flow between the first and second fluid delivery devices and flow from at least one of the first and second fluid delivery devices to the second fluid delivery channel. The fluid delivery channels will typically comprise sterilizable flexible polymer tubing; however rigid polymer or metal piping or other fluid conduit known in the art may also be used.

In many embodiments, the first and second fluid delivery devices comprise syringes which may be separate or attached to one another. In some embodiments the two syringes may have a unitary construction for example by being formed from one mold with a joining feature between the two (or other number) of syringes. In various embodiments utilizing syringes as the fluid delivery device, a drive means such as a syringe pump may be coupled to one or both syringes and is configured to a apply drive force to one or both of the syringes or other fluid delivery device. The syringe pump or other drive means can be configured to independently actuate each syringe or other fluid delivery device. Typically, one drive means will be coupled to both the first and second syringes, though in other embodiments, each syringe or other fluid delivery means can have its own drive means. In many embodiments, the syringe pump or other drive means are also operatively coupled to the controller such that fluid delivery from one or both syringes/fluid delivery devices are controlled by the controller.

According to some embodiments, the syringes can be manually filled by the user and the connected to the tubing or other fluid conduit comprising the fluid delivery channels. In alternative or additional embodiments, the syringes may be automatically filled automatic filling device which can in various embodiments be under control of the microprocessor or other embodiment of the controller

In alternative embodiments, one or more of the syringes and tubings used for the first and second fluid delivery channels can be replaced by one or more microfluidic chips. According to one embodiment, a microfluid chip can be configured as the fluid delivery device for both the plasmid and transfection reagent solutions, where it includes reservoir of each concentrated solution mounted or otherwise fluidically coupled to the chip via valves with a pneumatic pressure source coupled to the reservoirs to and a syringe or other fluid reservoir containing a buffer solution fluidically coupled to the chip and an outflow valve. The valves are desirably actuable by pneumatic or other actuating means known in the microfluidic arts which is desirable controlled by the controller or other control device allowing for control of each individual valve. The valves are coupled to one or more fluidic channels on the chip where the solutions can enter, mix and exist. In particular embodiments, one of the microfluidic channels comprises a mixing chamber or manifold where the incoming solutions and buffer can mix and then exit off the chip via the outflow valve which can be coupled to tubing or other fluidic channel coupled to the bioreactor vessel. The advantage of embodiments using microfluidic chip in these, and related configurations is that it optimizes mixing of the plasmid and transfection reagent reduce dead volumes allowing for faster delivery of the transfection solutions and reducing the delivery of other solutions (e.g., buffer solution) trapped in one or more tubing or other fluidic channels. In additional or alternative embodiments using a microfluid chip, the microfluidic chip can be configured to formulate the plasmid and transfection reagent solutions on demand (e.g., using concentrated reservoirs and buffer solutions as described in the previous embodiment) and then dispense them on demand to syringes for each separate solution. According to one or more embodiments, this process can be implemented through the use of a plurality of control valves (e.g., by pneumatic actuation) on the micro fluidic chip controlling the inflow and outflow of fluids from the microfluidic chip.

According to one or more embodiments, the controller corresponds to a processor such as microprocessor or other logic resources and includes a software module or other electronic instruction set operable on the processor for controlling one or more aspects of the transfection process. In particular embodiments, the software module is configured to control flow between the first and second fluid delivery devices and flow from one or both of the fluid delivery devices to at least one of the second fluid delivery channel or the bioreactor vessel. In use, such embodiments allow for the controlled mixing of the solutions within each fluid delivery device (e.g., the plasmid solution and the transfection agent solution) in order to achieve a desired output such as production of the transfection solution having one or more selected properties such as concentration of the transfection agent plasmid complexes.

The control valve can correspond to a variety of controllable valves known in art. Though in many embodiments the control valve will comprise a pinch valve which can be a solenoid based pinch valve or other controllable pinch valve known in the art. In some embodiments, the pinch valve can also be configured to be manually operated (either through the use of a partially or fully manual valve) allowing a user to open and close the valve, for example to allow mixing of the two solutions. In particular embodiments, the control valve may be a three-way valve to control flow from each of the fluid delivery devices and outflow to the second fluid delivery channel and the bioreactor vessel or other vessel or chamber. In some embodiments the control valve may be a wireless type valve configured to be controlled by an RF signal sent under control of the processor. In various embodiments of a wireless control valve the valve may be configured to be controlled using a BLUETOOTH™ or other wireless communication protocol known in the art.

In many embodiments the system also includes the bioreactor vessel which is directly or indirectly fluidically coupled to the second fluid delivery channel. In these and related embodiments including a bioreactor, the system may include other bioreactor components coupled to the vessel such as a headplate, dip tubes and the like. In many embodiments having a bioreactor vessel, the system also includes at least one sensor operatively coupled to the bioreactor vessel and for sensing one or more properties of the cell culture medium within the vessel including properties providing information on a biological condition of cells and/or cell culture medium. The at least one sensor is also desirably operably couple to the controller to allow for an output from the sensor to be used by the controller to control one or more aspects of the transfection process such as the preparation and addition of the transfection solution to the cell culture medium within the vessel. In various embodiments the at least one sensor may correspond to one or more of an optical, thermal, gas, pH or glucose or other chemical sensor. In particular embodiments, the sensor is configured to measure cell density or other related property/condition of the cell culture within the bioreactor vessel such as cell growth via rate of change of cell density. In these and related embodiment, the sensor may correspond to an optical sensor configured to measure turbidity or other optical property of the cell culture medium within the bioreactor vessel which provides information on the cell density within the vessel. Also in related embodiments, sensor 78 may also correspond to an electrical sensor 79′ configured to measure capacitance or other electrical property of the cell culture medium within the bioreactor vessel which provides information on the cell density including viable cell density within the vessel.

In various embodiments of using a sensor 78 for sensing properties of culture medium 76 (e.g., to determine a biological condition used for a trigger event), the sensor may be positioned within the bioreactor vessel for example by means of a dip tube placed within the vessel or alternatively it may be optically coupled to an exterior surface of the bioreactor such as bottom surface. In some embodiments, multiple sensors may be used and positioned at various locations within or on the bioreactor so as to obtain cell density measurements at multiple locations within or on the bioreactor. In use, such multiple sensor embodiments account for variations in cells density within the vessel which may occur due to incomplete mixing or other non-uniform property within the cell culture medium within the vessel and thus provide a more reliable measure of cell density used by a software module or other algorithm for controlling the transfection process including delivery of the transfection solution to the cell culture medium based on cell density or related property.

In many embodiments, the system for transfection may also include a mixing device that is fluidically coupled to at least one of the first and second delivery devices and the first fluidic channel. The mixing device is positioned and configured to mix the first and second solutions upon flow of the solutions by or through the mixing device. In various embodiments, the mixing device may be configured to do so passively by passive fluid flow through or by the mixing device or actively by movement or other action of the mixing device (e.g., rotational movement). In various passive embodiments, the mixing device may have various shapes internal and/or external that are configured to promote mixing including shapes configured to generate swirling and/or vortex flow of the two solutions.

For embodiments of the mixing device configured for internal mixing (e.g., by fluids passing through the mixing device) the mixing device will typically comprise two or more inflow ports positioned at or near one end of the mixing device for connection to incoming solutions to be mixed (e.g., the plasmid solution and the transfection agent solution), an interior mixing chamber where mixing occurs and an outflow port at or near an opposite end of the mixing device. The length of the mixing device and/or mixing chamber can also be selected based on flow rates through the mixing device so as to control the residence time of the solutions to be mixed within mixing chamber as to achieve a desired amount of mixing. In various embodiments the length of the mixing device can range from about 5 to 50 cms depending on the flow rates of the two or more solutions passing through the mixing chamber.

Desirably, the interior mixing chamber has one or more of a shape, size and other characteristic to produce swirling or other flow characteristic for mixing of incoming solutions. In particular embodiments, the mixing chamber of the mixer has a helical shape with two or more in-flow ports on one side of the helix and an out-flow port on the side of the helix. Also in many embodiments, the shape (e.g., diameter and shape of the helix) of the internal chambers is configured relative to anticipated flow rates such that shear forces imparted by fluid passing through the internal shape is below that which would cause damage to the plasmid DNA (or other extrachromosomal DNA) and/or the transfection agent plasmid complexes. For active mixing embodiments, the mixing device may have one or more of a rounded cylindrical shape (e.g., such as used for magnetic stir bars) and/or a propeller shape.

In addition to other embodiments described herein, one embodiment of a method of using an embodiment of the system for transfection comprises the following. An operator fills the two syringes with the reagents for the transfection process including plasmids and transfection reagent, typically PEI (or other lipid or polymer-based transfection reagents). These two reagents can be diluted in cell culture media and dispensed into the syringes in a relatively time-insensitive manner, hours in advance of the transfection. Optionally, filling can be performed or aided by an automatic syringe filling system or a microfluid chip. The filled syringes are then loaded into a pumping system contained or otherwise coupled to the bioreactor.

Once the syringes are loaded, the rest of the transfection process is controlled in an automated and pre-programmed way according to parameters chosen by the operator and/or a remote user. Upon a trigger event which can be a specified time, or an input from a specific sensor (e.g., which senses cell density, metabolic activity, and the like) in or otherwise operably coupled to the bioreactor, the transfection process will begin. The first step is mixing, in which one syringe plunger is depressed while the other is pulled out (which will typically be done by dual action multi-syringe syringe pump. Combined with a pinch valve blocking the path to the bioreactor, this will cause the two reagents to mix together. The mixing rate and shear force experienced by the reagents are controlled by the pumping rate between the two syringes and thus are highly repeatable.

After mixing, the combined reagents will reside in one of the two syringes for the programmed transfection time. Periodic mixing in a user-defined schedule is also enabled by this system if desired, to the benefit of achieving the most effective transfection complexes possible.

Finally, the injection of the transfection reagents is then triggered by one of the above factors (e.g., cell density, time, or other sensed condition) and the vectors are delivered to the cell culture by opening the pinch valve and depressing the full syringe. The pumping speed, shear forces, and timing are all automated (e.g., using a microprocessor or other programmable controller described herein) and thus repeatable, characterized, and consistent.

Embodiments of the above methods and systems for transfection provide a number of benefits. First, embodiments of the systems and methods for automating the transfection of packaging cells (e.g., mammalian cells) for the production of viral vectors increase the reproducibility and consistency of the process and the resulting production of virus particles (e.g., virions) by the packaging cells. This in turn increases the reproducibility and consistency of one or more of the viral production parameters described herein. Also, they improve both the accuracy and precision of the solution handling steps during preparation and delivery of the transfection solution in turn leading to improved effectiveness of transfection and improved accuracy and precision of one or more of the viral production parameters discussed herein. Further, they reduce the time and labor involved in performing transient transfection including the preparatory steps of mixing and adding the transfection solutions to the cell culture media. Additionally, embodiments of automated systems for transfection including integrated data management collection and analysis systems and tools provide faster and more accurate data analysis and facilitating easier data management and interpretation. Still other benefits of embodiments are contemplated and/or will be apparent to those skilled in the art.

Various embodiments provide systems and methods for transfecting mammalian or other eukaryotic cells to produce a selected virus wherein the system is configured to automate and/or control one or more aspects of the process. Such selected viruses may correspond to various genetically engineered viral vectors for use in various research and medical applications including gene therapy, oncology (as an oncolytic virus) and DNA vaccination.

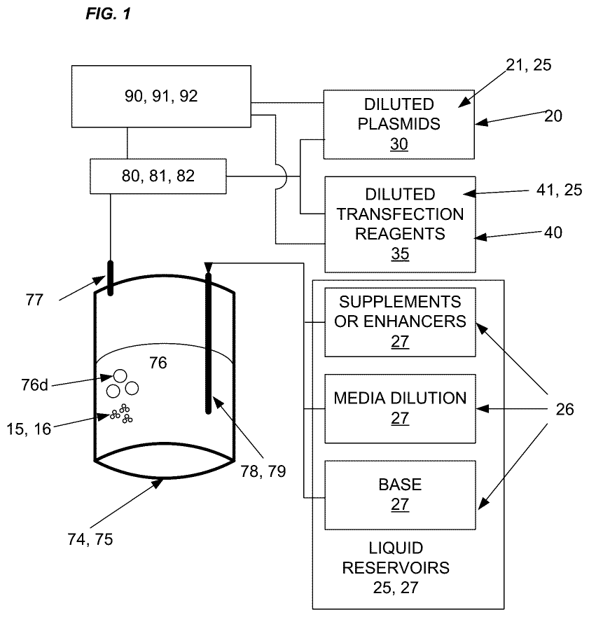

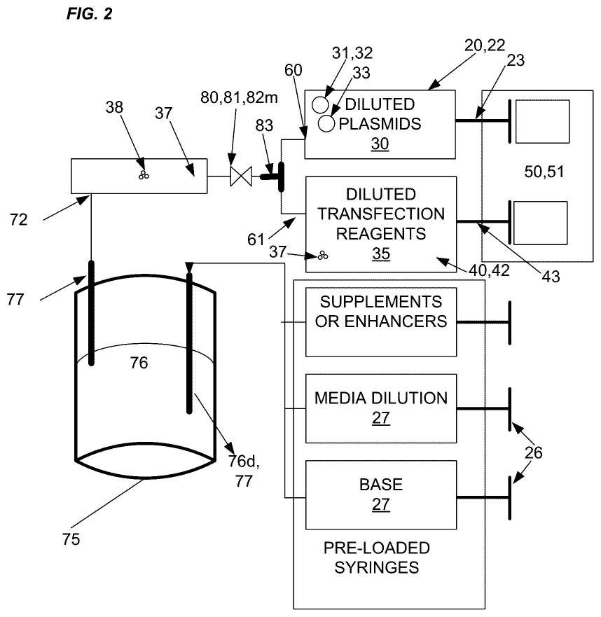

Referring now to FIGS. 1-3, one embodiment of such a transfection system 10 to generate a selected virus 15 comprises a first fluid delivery device 20 configured to contain and deliver a first solution 30 comprising extrachromosomal DNA molecules 31 such as plasmids 32 encoding the virus and a second fluid delivery device 40 configured to contain and deliver a second solution 35 comprising a transfection agent 36. For ease of discussion, first and second solutions 30 and 35 are sometimes referred to herein as plasmid solution 30 and transfection agent solution 35. Fluid delivery devices 20 and 40 have fluid chambers 21 and 41 for storing and mixing solutions 30 and 35. Chambers 21 and 41 can also function as reservoirs 25 of solutions 30 and 35 to be delivered to one or more bioreactor vessels. System 10 can also include other reservoirs 26 for other solutions 27 (e.g., media supplements or enhancers, diluted culture media or a base solution) to be delivered to one or more bioreactor vessels. A first fluid delivery channel 60 is fluidically coupled to the first and second fluid delivery devices 20 and 40. Also, at least one control valve 80 is fluidically coupled to at least one of the first and second fluid delivery devices 20 and 40 and the first fluidic delivery channel 60. The control valve 80 is configured to control flow between the first and second fluid delivery devices 20 and 40 and outward flow from one or both of the fluid deliver devices to a bioreactor vessel 75 or other component in various embodiments of system 10 (e.g., a mixing device, or incubation chamber) via a second or another fluid delivery channel 70. In many embodiments, the control valve 80 is operably coupled to a controller 90 which is configured to control valve 80 so as control flow between the first and second fluid deliver devices 20 and 40 and flow from at least one of the first and second chambers to the second fluid delivery channel. The fluid delivery channels 60 and 70 will typically comprise sterile flexible polymer tubing 61, 71 where the polymer materials are selected for their ability to be sterilized (e.g., by autoclave, e-beam or ethylene oxide). However, in alternative embodiments, rigid plastic, metal piping or other fluid conduit material known in the art may also be used.

In many embodiments, the first and second fluid delivery devices 20 and 40 comprise syringes 22 and 42 having plungers 23 and 43. In various embodiments syringes 22 may be separate or attached to one another. In some embodiments the two syringes 22 and 42 may have a unitary construction, for example, by being formed from one mold with a joining feature between the two (or other number) of syringes. Configurations of syringes 22 and 42 having a unitary construction can in one or more embodiments be configured to be loaded and be filled with an automatic filling device.

In various embodiments utilizing syringes as fluid delivery devices 20 and 40, a drive or actuation means 50 such as a syringe pump 51 may be coupled to one or both syringes 22 and 42 and is configured to apply a drive force to one or both of the syringes. More specifically, embodiments of pump or other drive means 50 can be configured to engage syringe plungers 23 and 43 to both push or pull the syringe plunger to push out or pull in fluid to each syringe. Pump 51 or means 50 can further configured to eject or draw in precise volumes of fluid through the use of stepper motors and associated stepper motor controller and control logic. Typically, one syringe pump 51 or other drive means 50 will be coupled to both the first and second syringes, though in other embodiments, each syringe or other fluid delivery device can have its own pump 51 or other drive means 50 which can be independently actuated. Embodiments having one syringe pump 51, can also be configured to independently actuate each syringe. In addition to independent actuation, syringe pump 51 can have a variety of modes of operation adapted to a particular step in the transfection process. For example, the one syringe pump 51 can in various embodiments be configured (e.g., by a software module 96 or other module 91) to reciprocally actuate syringe 22 and 42 including syringe plungers 23 and 43 such that when one plunger is being advanced to push out fluid from that syringe while the other plunger is being withdrawn to pull in fluid into that syringe. Such reciprocal motion embodiments can be employed to circulate fluid back and forth between syringes 22 and 42 so as mix plasmid and transfection agent solutions 30 and 35 (or other solution) to form a transfection solution 37 containing plasmid-transfection agent complexes 38 (also referred to herein as transfection complexes, transfection agent plasmid complexes or liposome plasmid complexes) or other solution in the course of the transfection process. In many embodiments, the syringe pump 51 is operatively coupled to controller 90 such that fluid delivery from one or both syringes/is controlled by the controller. One example of such control being for the reciprocal motion mode just described.

In one or more embodiments, system 10 can include a chamber 72 positioned down-stream from syringes 22 and 42 where the mixed solutions 30 and 35 making up transfection solution 37 can be stored and incubated to form and/or further the formation of transfection complexes 38 before transfection solution 37 is delivered to bioreactor 75. In these and related embodiments, chamber 72 can thus function as an incubation chamber 72 for incubation of transfection solution 37.

According to some embodiments, syringes 22 and 42 can be manually filled by the user and then connected to the tubing or other fluid conduit comprising the fluid delivery channels. In alternative or additional embodiments, syringes 22 and 42 may be automatically filled by an automatic filling device (described herein) which can in various embodiments be under control of the microprocessor or other embodiment of controller 90.

According to one or more embodiments, the controller 90 corresponds to a processor such as a microprocessor or other logic resources for executing electronic instructions. In many embodiments processor 90 includes at least one software module 91 or other electronic instruction set operable on the processor for controlling one or more aspects of the transfection process which can include one or more of the following: mixing and delivery of transfection solution 37 to bioreactor vessel 75, delivery of other solutions (e.g., cell culture media) to vessel 75, control of environmental conditions in vessel 75 and cell culture media 76, the inoculation and growth of cells 76s within media 76 and the production of virus 15 by cells 76c within media 76. In many embodiments, one or more software modules 91 for controlling various aspects of the transfection process can reside in the cloud 100 for example, in cloud data storage 101, as part of a cloud-based architecture 102 which can link various software controllable components and/or input devices of system 10 (e.g., valves, pumps, sensors, barcode scanners and the like) so as to form a cloud-based transfection system 110.

In many embodiments, software module 91 can include or otherwise correspond to a number of submodules 92 for controlling specific aspects of transfection or related processes. Such submodules 92 can include a recipe control module 93 containing various logic process conditions and other parameters for a recipe for producing a particular viral vector other virus; a transfection trigger module 94 containing logic for initiating the transfection process in response to one or more trigger events (e.g., cell density, culture media condition or time) based on sensor data; an environment control module 95 containing logic for feedback control of one or more environmental conditions (e.g., pH, oxygen content, temperature,) within vessel 75 and media 76 using sensor data; and a syringe module 96 including logic to control flow between the first and second delivery syringes 22 and 42 (or other fluid delivery device the 20 and 40) and flow from one or both syringes to at least one of the second fluid delivery channel or the bioreactor vessel 75. Embodiments of each of the aforementioned modules provide a number of benefits. For example, recipe module 93 allows all the process conditions for generation of a particular virus 15 such as a genetically engineered viral vector to be stored rather than hand entered improving batch to batch consistency and reproducibility. The trigger module 94 allows the transfection process to be precisely triggered for optimizing transfection efficiency and other various viral production parameters described herein. The environmental control module 95 allows precise control of environmental conditions within vessel 75 and media 76 so as to optimize packaging cell growth within the media and in turn the production for virus by the cells and resulting viral production parameters. Finally, the syringe control module 96 allows for the controlled mixing of the solutions within each syringe (e.g., the plasmid solution and the transfection agent solutions 30 and 35) in order to achieve a desired output such as production of a transfection solution 37 having one or more selected properties such as concentration of the transfection agent plasmid complexes 38 and uniform distribution of the complexes within the solution. This in turn leads to improved/optimized transfection, virus production and resulting viral production parameters.

Control valve(s) 80 can correspond to a variety of controllable valves known in art. In preferred embodiments, control valve 80 will is a pinch valve 81 configured such that that valve need not come into contact with the inner portion of tubing 61, 71, or other tubings used in system 10 so that actuation of the valve does not compromise the sterility of the tubing. In various embodiments, pinch valve 81 corresponds to a solenoid-based pinch valve or other controllable pinch valve known in the art. In some embodiments, the pinch valve 81 can also be configured to be manually operated valve 81m (either through the use of a partially or fully manual valve) allowing a user to open and close the valve for example to allow mixing of solutions 30 and 35. In particular embodiments, control valve 80 may be a three-way valve 83 configured to control flow from each of the fluid delivery devices and outflow to the second fluid delivery channel and the bioreactor vessel 75 or other vessel or chamber in various embodiments of system 10. In some embodiments, the control valve may be a wireless type control valve 82 configured to be controlled by an RF signal sent under control of controller/processor 90. In various embodiments of a wireless control valve, the valve may be configured to be controlled using a BLUETOOTH™ or other wireless communication protocol known in the art.

In many embodiments transfection system 10 also includes the bioreactor vessel 75 which may be directly or indirectly fluidically coupled to the second fluid delivery channel 70 so as to receive transfection solution 37. Bioreactor vessel 75 is configured to contain and incubate cell culture medium 76 containing cells 76c to facilitate cell growth, transfection and virus production. In these and related embodiments including a bioreactor vessel 75, the system may include other bioreactor components 77 coupled to the vessel such as a dip tubes, headspace nozzle, headplate, and the like. In many embodiments having a bioreactor vessel 75, the system also includes at least one sensor 78 operatively coupled to the bioreactor vessel for sensing one or more properties of the cell culture medium within the vessel including properties providing information on a biological condition of cells and/or cell culture medium. The at least one sensor 78 is also desirably operably couple to the controller 90 to allow for an output 780 from the sensor to be used by the controller to control one or more aspects of the transfection process such as the preparation and addition of the transfection solution to the cell culture medium 76 within vessel 75. In various embodiments, the at least one sensor may correspond to one or more of an optical, thermal, gas, pH or glucose or other chemical sensor. In particular embodiments, the sensor is configured to measure cell density or other related property/condition of the cell culture within the bioreactor vessel such as cell growth via rate of change of cell density. In these and related embodiments, the sensor 78 may correspond to an optical sensor 79 configured to measure turbidity or other optical property of the cell culture medium within the bioreactor vessel which provides information on the cell density within the vessel. In these and related embodiments, the sensor may be positioned within the bioreactor vessel 75 for example by means of a dip tube 77d placed within the vessel or alternatively, it may be optically coupled to an exterior surface of the bioreactor vessel such as bottom surface. In some embodiments multiple sensors 78 may be used and positioned at various locations within or on the bioreactor vessel 75 so as to obtain cell density measurements at multiple locations within or on the vessel 75. In use, such multiple sensor embodiments provide the benefit of accounting for variations in cell density within the vessel which may occur due to incomplete mixing or other non-uniform property within the cell culture medium within the vessel This in turn provides a more reliable measure of cell density used by a software module 91 or other algorithm for controlling the transfection process including delivery of the transfection solution to the cell culture medium based on cell density or related property.

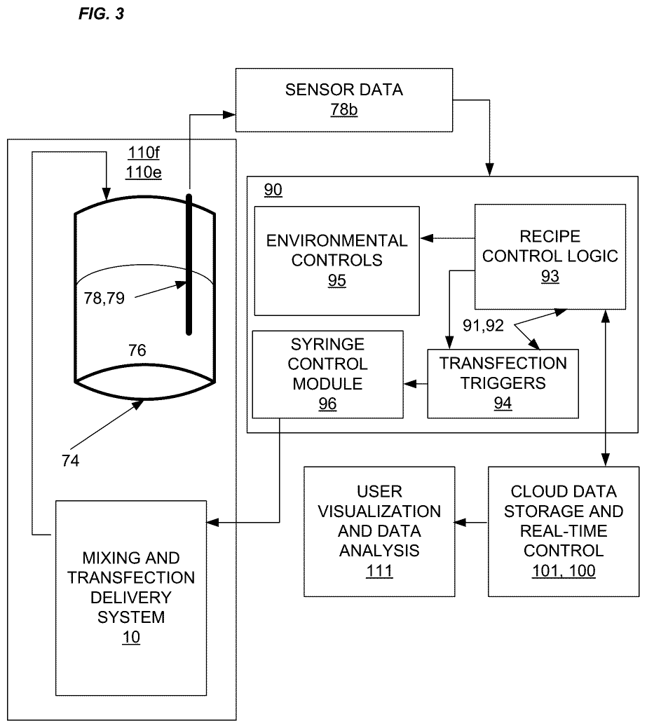

Referring now specifically to FIG. 3, a description of an embodiment of a cloud-based transfection/bioreactor system 110 employing a cloud-based architecture 102 will now be provided. The system can include both firmware and software feedback control subsystems 110h and 110s for regulating one or more aspects of the transfection process. The former includes firmware embedded in components of electromechanical systems 110e. The specific components of the system can include a mixing and transfection delivery system 105, a bioreactor 74 including a bioreactor vessel 75 having one or more sensors 78, a processor 90 operatively coupled to one or more of the bioreactor 74, bioreactor vessel 75 or sensors 78; the cloud 100 operatively coupled to processor 90; and user visualization and data analysis and input resources 111 operatively coupled to the cloud. Processor 90 includes one or more software modules 92 for controlling one or more aspects of the transfection process including, for example recipe control module 93, transfection trigger module 94, environmental control module 95 and syringe control module 96. Cloud 100 includes cloud data storage 101 allowing for storage of data received from sensors 78 and/or component of transfection system 105, as well as storage of one or more modules 92 (e.g., recipe control module 93) which can be uploaded by a user via resources 111. User visualization, data analysis and input resources 111 may correspond to a remote computer or smart phone connected to the cloud via the internet and allow the user to view and analyze real time data received from system 110 including for example data from one or sensors 78 as well as upload data and/or software modules, for example, recipe control module 93 containing process parameters for a transfection and virus production run. In practice, embodiments of cloud-based system 110 allow for the following beneficial uses. Process parameters for a transfection and virus production run via can be programmed in advance by a user remotely and transmitted through the cloud to the relevant bioreactor system. Real-time feedback and data can then be transmitted back up to the cloud for visualization by the user allowing for real time modification of the production run, if needed. Also, sensors can provide feedback to not only control the bioreactor but can also be utilized for the transfection trigger process, lending advantages to this fully integrated system over standalone methods of automation.

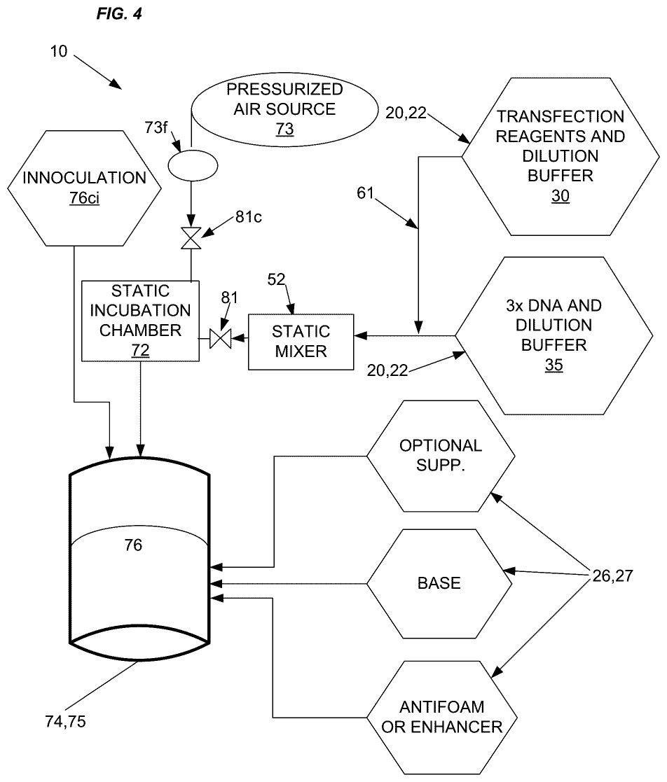

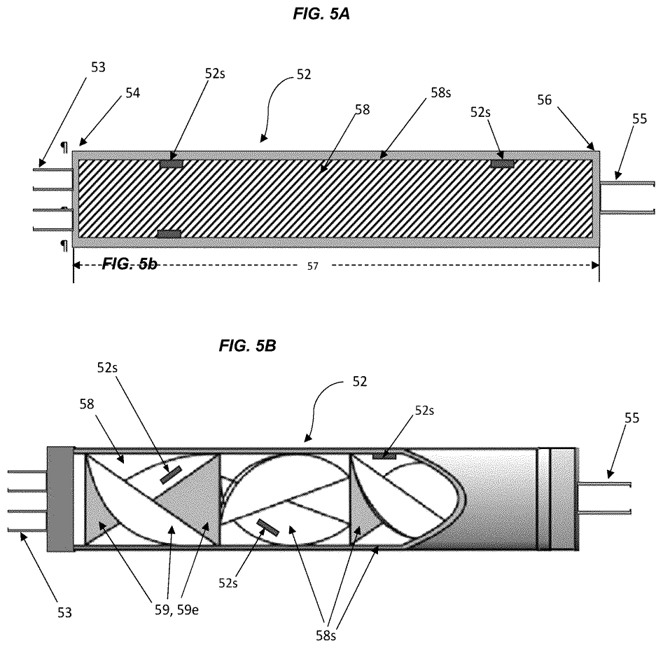

Referring now to FIGS. 4 and 5a-5b, in many embodiments, transfection system 10 may also include a mixing device 52 (also referred to as a mixer 52) that is fluidically coupled to at least one of the first and second delivery devices 20 and 40 and the first fluidic channel 60. Mixing device 52 is positioned and configured to mix the first and second solutions 30 and 35 upon flow of the solutions by or through the mixing device. In various embodiments, mixing device 52 may be configured to do so passively by passive fluid flow through or by the mixing device or actively by movement or other action of the mixing device (e.g., rotational movement). In various passive embodiments, mixing device 52 may have various shapes internal and/or external that are configured to promote mixing including shapes configured to generate swirling and/or a vortex flow of the two solutions.

For embodiments of mixing device 52 configured for internal mixing (e.g., by fluids passing through the mixing device), the mixing device will typically comprise two or more inflow ports 53 positioned at or near one end 54 of the mixing device for connection to incoming solutions to be mixed (e.g., the plasmid solution and the transfection agent solution), an interior mixing chamber 58 (also referred to as a mixing chamber) where mixing occurs and an outflow port 55 at or near an opposite end 56 of the mixing device. The length 57 of mixing device 52 and/or mixing chamber 58 can also be selected based on flow rates through the mixing device so as to control the residence time of the solutions to be mixed within mixing chamber 58 so as to achieve a desired amount of mixing. In various embodiments, length 57 can range from about 5 to 50 cms depending on the flow rates of the two or more solutions passing through the mixing chamber.

Desirably, the mixing chamber 58 has one or more of a shape, size and other characteristic to produce swirling or other flow characteristic for mixing of incoming solutions. In particular embodiments, the internal mixing chamber of the mixing device comprises a helix 59 and/or helical elements 59e with two or more in-flow ports 52 on one side of the helix 59 and an out-flow port on the other side of the helix. Also in many embodiments, the shape 58s (e.g., diameter and shape of the helix) of the mixing chamber 58 is configured relative to anticipated flow rates such that shear forces and/or strains rates imparted by fluid passing through the mixing chamber are below that which would cause damage (i.e., by the shear forces or strain rates) to the plasmid DNA 31 (or other extrachromosomal DNA) within solution 30 and/or the transfection agent plasmid transfection complexes 38 within solution 37. In particular embodiments, the shape 58s and flow rates through the mixing chamber 58 are configured to maintain the shear force in the mixing chamber below about 40 dynes/cm², more preferably below about 16 dynes/cm² and still more preferably below about 5 dynes/cm². In related embodiments, the shape 58s and flow rates through chamber 58 are configured to maintain strain rates below about 1.5×104 sec⁻¹ and more preferably below about 7×103 sec⁻¹. In one or more embodiments, the maintenance of flow rates through the mixing chamber 58 below that which would result in the above shear forces and/or strain rates can be facilitated by the placement of one or more sensors 52s within mixing chamber 58 or other location within mixing device 52 where such sensors are configure to measure flow rates and/or shear forces within mixing chamber 58 and/or other location within mixing device 52. Desirably, sensors 52s are operatively coupled to controller 90 and/or syringe pump 51 to provide feedback to these devices on flow rates and/or shear stress such that a feedback control algorithm (e.g., using, P, PI or PID control methods known in art) can be used to maintain flow rates through the mixing chamber 58 below that which would cause damage to plasmid DNA 31 (or other extrachromosomal DNA) within solution 30 and/or the transfection agent plasmid transfection complexes 38 within solution 37. In particular embodiments, such feedback control algorithms can be incorporated into software module 96 for controlling flow from syringe pump 51 or other syringe drive means 50.

Use of mixing device 52 in one or more embodiments provides several benefits. First, it provides improved and more consistent mixing of plasmid and transfection agents solutions 20 and 30 thus improving the consistency of transfection solution 37. Another benefit is that an operator need not meter out exact volumes of solutions 30 and 35 into syringes 22 and 42 in order to allow for proper mixing of the solutions back and forth between the two syringes or other fluid devices 20 and 40. The allowance for inexact metering of the solutions in turn saves time in the performance of the transfection process.

For active mixing embodiments, mixing device 52 may have one or more of a rounded cylindrical shape (e.g., such as used for magnetic stir bars) and/or a propeller shape (with two, three, four or more blades) and may include one or more magnetics for magnetically induced rotation or other movement of the mixing device. Again, whatever its shape, the active embodiments of the mixing device 52 are desirably configured to maintain the fluid shear stress induced by its movement (e.g., rotational or otherwise) below that which would cause shear damage to the plasmid DNA (or other extrachromosomal DNA) within plasmid solution 30 and/or transfection solution 37. This can be facilitated by the placement of one or more sensors 52s at one or more locations on the surface of active mixer 58 for sensing flow rates and/or shear forces resulting from movement (e.g., rotation) of mixing device 58.

In many embodiments of system 10 having a mixing device 52, the system can also include a static incubation chamber 72 positioned downstream from mixing device 52 for incubation of the mixed transfection solution 37. Typically, there will be a pinch valve 81 for example, pinch clamp valve 81c, positioned between mixing device 52 and incubation chamber 72 to control the flow of liquid between the device 52 and chamber 72. Chamber 72 is fluidically coupled to bioreactor 75 by means of sterile tubing 71 or other sterile fluid connection known in the art. In these and related embodiments, an air pressure source 73 can be fluidically coupled to incubation chamber 72 to provide a driving pressure for advancing mixed transfection solution 37 to bioreactor 75 with a sterile filter 73f positioned between the pressure source 73 and chamber 72, filter 73f configured to filter out viruses, bacteria, spores or other microbe or microbe sized particles. Such embodiments will also typically include a pinch valve 81, such as a pinch clamp valve 81c positioned between the chamber 72 and pressure source 73 to control the application of pressure from source 73 to chamber 72 and thus the flow of transfection solution 37 from chamber 72 to bioreactor vessel 75.

Also, in one more embodiments of system 10 employing mixing device 52 and incubator 72, bioreactor vessel 75 including cell culture media 76 can be manually inoculated with an inoculum 76ci (also referred to as inoculation 76ci) of packaging cells for growth in the cell culture media and subsequent transfection with plasmid or other extrachromosomal DNA 31.

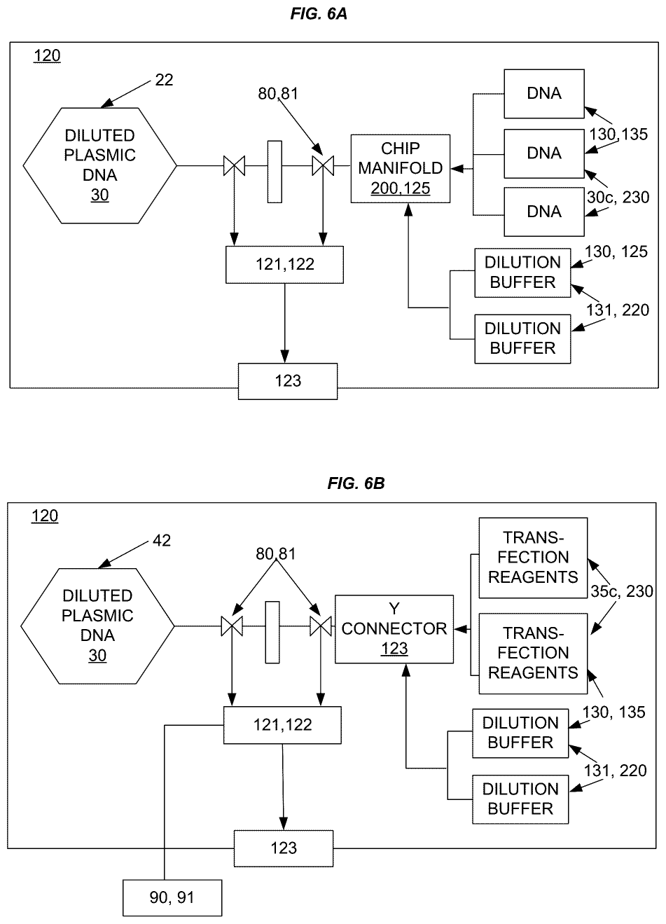

Referring now to FIGS. 6a and 6b, in various embodiments one or both of syringe 22 and 42 can be filled with their respective solutions (e.g., plasmid solution 30 and transfection agent solution 35) using an auto-filling device 120. Device 120 is configured to be loaded and engage with syringes 130 (or other reservoir 135) containing stock solutions 220 used to prepare each final solution 30 and 35. Once the syringe(s) is loaded, device 120 then engages the syringe(s) to pull out a metered volume of the respective stock solution, mix them and then inject the final mixed solution into one of syringes 22 and 42. One or more of these operations can be performed under software control, for example using one or more software modules 121 operative on a processor 122 contained within device 120 and/or by modules 91 operative on processor 90 for embodiments of system 10 and/or 100 where processor 90 is operatively coupled to device 120. In one or more embodiments, auto-filling device 120 may correspond to those described in U.S. Provisional Patent Application Ser. No. 63/552,997, filed on Feb. 13, 2024 entitled: “Apparatus, System, And Method For Automated Filling Of Syringes” the contents of which are incorporated herein for all purposes.

For preparation of plasmid solution 30 depicted in FIG. 6a, one or more syringes 130 (or other reservoirs 135) containing concentrated plasmids solutions 30c and buffer solutions 131 are loaded into the auto filler 120 and the user then presses an actuation button 123 on the auto-filler which can be analogue or on a touch screen. Upon pressing button 123, the stock solutions are pulled from syringes 130 and advanced through a microfluidic chip 200 (described below) or miniature manifold 125 where the solutions mix. The user can then connect plasmid solution syringe 22 into to the chip or manifold and through manual or software actuation of pinch valve 81, the plasmid solution is injected by the auto filler into syringe 22. A similar operation can be performed for the preparation of the transfection agent solution 35 as depicted in FIG. 6. In this case a Y-connector 124 can be used to connect lines coming from syringes 130 (or other reservoir 135) containing concentrated transfection agents solution 35c and buffer solution 131.

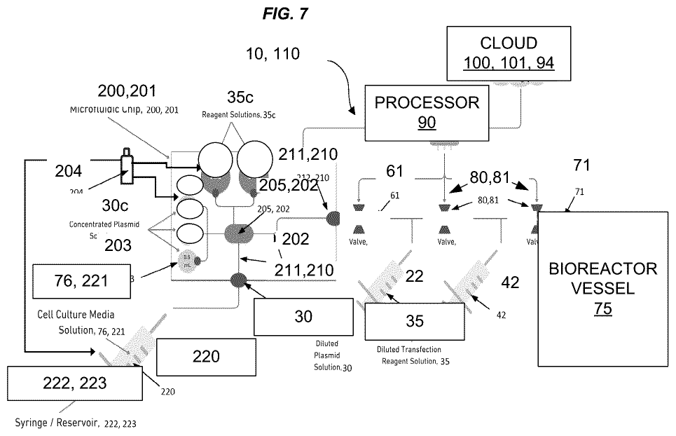

Referring now to FIG. 7, in alternative embodiments, one or more of the syringes 22, 42 and tubings 61 used for the first and second fluid delivery channels 60 can be replaced by one or more microfluidic chips 200 configured to perform one or more steps in the preparation and delivery of solutions used in the transfection process (e.g., solutions 30 and 35). In such embodiments, chip 200 can be operatively coupled to processor 90, for control of one or more operations on or by chip 200 (e.g., inflow, outflow and mixing of solutions). Such control can be implemented through the use of one more software modules 91 operative on the processor, such modules including, for example, a recipe control module 93 which can in various embodiments, be downloaded from the cloud 100 including, for example, from cloud data storage 101 in embodiments of a cloud-based transfection system 110.

According to one embodiment, microfluid chip 200 can be configured as an on-demand solution preparation device 201, also referred to as chip 201, for plasmid and transfection reagent solutions 30 and 35. In such embodiments, chip 201 typically includes fluidic channels 202, valves 210 and reservoirs 203 of concentrated plasmid and transfection reagent solutions 30c and 35c mounted on or otherwise fluidically coupled to the chip via valves 210. One or more stock solutions 220 (e.g., media solution 76 or a dilution buffer solution 221) used to formulate each solution 30 and 35 can be supplied from a syringe 222 (or other external fluid reservoir 223) coupled to the chip through an inflow valve 211. The formulated solutions flow off the chip through outflow valve 212. A pneumatic or other pressure source 204 can be coupled to the reservoirs 203 and/or valves 210 so as to provide pressure for advancing fluid from the reservoirs 203 through valves 210 and into fluidic channels 202.

Valves 210, 211, and 212 can be configured to be actuable by pneumatic or other actuating means known in the microfluidic and are desirably controlled by controller 90 or other control device allowing for control of each individual valve. For pneumatic control embodiments, pressure source 204 can also be coupled to one or more valves 210, 211, and 212 so as to actuate each valve to open or close the valve. Other actuation means for the valves can include electro-kinetic (e.g., piezo-electric), or an external force application means, for example by means of a solenoid.

Valves 210, 211 and 212 are coupled to one or more fluidic channels 204 on chip 201 where solutions can enter, mix, and exit. In particular embodiments, one of the microfluidic channels 202 may comprise or be coupled to a mixing chamber or manifold 205 where the incoming solutions can mix, and then exit off the chip via the outflow valve 211 which can be coupled to tubings 61, 71 or other fluidic channel for ultimate delivery to bioreactor vessel 75.

In some embodiments chip, 201 can prepare and then mix solutions 30 and 35 to form transfection solution 37 which can then be directly delivered to bioreactor vessel 75 valve 80 without being stored in either of syringes 22 or 42 (or other fluid delivery devices 20 and 40). In these and related embodiments syringes 22 or 44 need not be included in system 10.

In other embodiments, chip 201 can be fluidically coupled to syringes 22 and 42 for delivery of prepared solutions 30 and 35 to the respective syringe. In these and related embodiments, once the prepared solutions 30 and 35 exit chip via valve 211 they can then be directed to flow into either syringe 22 or 42 by valves 80, 81 (which can be under control of processor 90) for subsequent mixing and flow into bioreactor vessel 75.

The advantage of embodiments using a microfluidic chip 200, 201 in these and related configurations is that it allows for on-demand formulation of plasmid and transfection agent solutions 30 and 35 reducing the time and necessity of preparing and storing these solutions in advance of the transfection process. Use of one or more microfluidic chips 200 and 201 also optimizes mixing of solutions 30 and 35 and reduces dead volumes allowing for faster delivery of the solutions and reduces the delivery of any unwanted solutions trapped in one or more tubings 61 and 71 or other fluidic channels.

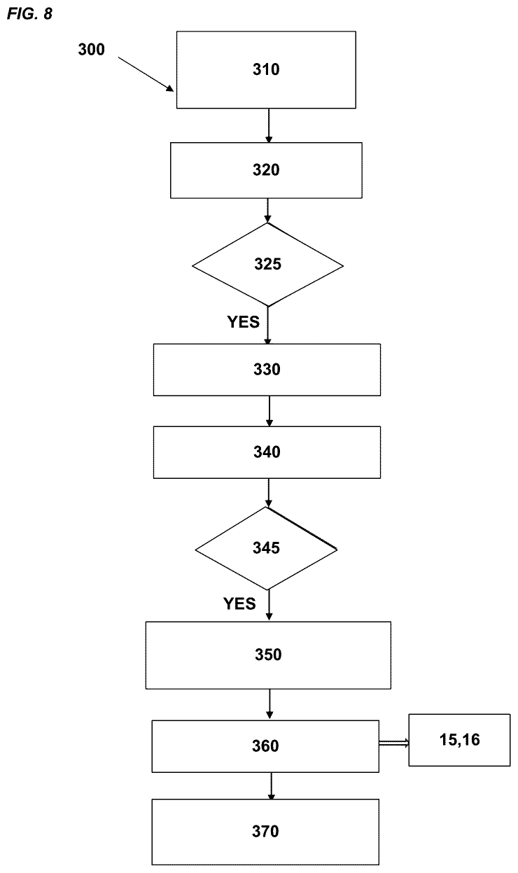

Embodiments also provide methods for the transfection of mammalian or other cells to produce a selected virus. The selected virus in many embodiments may correspond to a viral vector configured to deliver genetic coding material to target cells within a human or other mammal or organism. Referring now to FIG. 8, one embodiment of a such a transfection method 300 comprises providing, in step 310, a first solution 30 comprising a plasmid or other extrachromosomal DNA encoding a virus and in step 320 providing a second solution 35 comprising a transfection agent wherein the first and second solutions are kept separate. Then, the two solutions are mixed in step 330 to produce a transfection solution 37 to be delivered to a cell culture medium 76 for transfection of cells 76c in the medium with the extrachromosomal DNA 31 wherein the mixing is initiated responsive to a trigger event at 335 (herein trigger event 335, which can be the occurrence and/or detection of an event). The transfection solution is then incubated to form transfection complexes in step 340. Then, responsive to the same 335 or a different/second trigger event 345, the transfection solution is delivered in a step 350 to the cell culture medium 76 in vessel 75 to transfect the cells 76c in the medium to produce the selected virus 15 in step 360 wherein a viral production parameter 17 resulting from transfection of the cells is optimized (indicated by step 370) in at least by initiating the mixing of the first and second solutions responsive to the first trigger event 335. The production parameter may also be optimized by delivering the transfection solution responsive to the second trigger event 345.

According to one or more embodiments, the viral production parameter 17 may correspond to one or more of a viral concentration, a batch-to-batch variation of viral concentration, an amount of variation in the genetic composition of a population of viral particles from a production batch, an average virus infectivity of a viral production batch. For embodiments utilizing viral concentration or derivative property as the viral production parameter 17, the concentration may correspond to physical titer or functional titer, the former measured in gc or vg/ml and the latter in transductions unit (TU) per ml, plaque-forming units per mL (pfu/mL), or infectious units per mL (ifu/mL), depending on the viral vector. For embodiments where the production parameter 17 is a batch-to-batch variation in viral concentration (either physical titer or functional titer), in one or more embodiments, the batch-to-batch variation (as measured by coefficient of variation) can be less than ±20%, more preferably less than ±10% and still more preferably ±5%. In particular embodiments, these values can be achieved by using measurements of cell density of the cell culture media 76 as the trigger event for the mixing and delivery of transfection solution 37 to the cell culture media. Even lower values can be achieved by combining measurement of cell density with other measurements of the cell culture media predictive of optimal conditions for the cells being transfected with viral encoding DNA 31 and producing virus. Such conditions can include one or more of pH, oxygen concentration and CO2 concentration.

Typically, the virus 15 will comprise a genetically modified virus 16 which is engineered to deliver one or more genes or other genetic material to target cells in a human, mammal or other animal or organism upon infection of the target cells with the virus. Such viruses are also referred herein as viral vectors. In many embodiments, the one or more genes are selected and/or engineered to treat a genetic disease or condition such as vision loss, sickle cell anemia, hemophilia, cystic fibrosis, or various glycogen storage disorders such as Pompe Disease. In other embodiments, the genetically engineered virus 16 may comprise an oncolytic virus for the treatment of solid tumors or other forms of cancer whereby the virus infects the tumor either killing the tumor cells or drawing in immune cells to do so. In various embodiments, the genetically modified virus 16 correspond to one or more of an adenovirus, an adeno-associated virus, a lentivirus, a retrovirus, an onco-retrovirus or other viruses known in the gene therapy, vaccine and viral transfection arts.

In many embodiments, the extrachromosomal DNA molecules 31 correspond to plasmids 32, however other forms of extrachromosomal DNA known in the art may also be used. In some embodiments, all the plasmids in solution may essentially be identical and carry the same DNA encoding the same genes while in others the first, solution also described as the plasmid solution 30 may include one more different plasmids 33 each including DNA for a different gene or group of genes. In specific embodiments, the plasmid solution 30 may comprise between 2 to 20 different plasmids 33 having different DNA encoding different genes and/or viral genomes.

In various embodiments, the transfection agent 36 may correspond to lipid and/or liposomes selected to increase the efficiency of transfection of the selected mammalian or other eukaryotic cells with the plasmid or other extrachromosomal DNA. In particular embodiments, the transfection agent 36 corresponds to lipofectamine which contains cationic liposomes. These liposomes form complexes with the plasmid (or other extrachromosomal DNA) wherein the plasmids are entrapped within the liposome. The liposome plasmid complexes 38 (also referred to herein as plasmid-transfection agent complexes, transfection complexes, or transfection agent plasmid complexes) then fuse and cross cell wall of the cell to be transfected allowing the plasmid DNA cargo to get into the cell cytoplasm for expression within the cell. The use of other liposomes including cationic liposomes known in the art as transfection agents is also contemplated. In use, these or other transfection agents 36 serve to increase the efficiency of transfection of the target cells with the selected plasmid or other extrachromosomal DNA. In particular embodiments, one or more of the transfection agent and its concentration within the transfection solution 37 can be selected and/or titrated to achieve a desired transfection efficiency (e.g., 59, 60, 70, 80%, etc.) depending on one more of the plasmid DNA encoding the virus, the packaging cell line and the packaging cell culture conditions. In one or more embodiments, the titration levels of the transfection agent 36 can be stored in recipe control module 93.

In various embodiments, the trigger events 325 or 345 may correspond to a variety of events related to the cell culture medium 76 and/or the process for preparing and incubating the cell culture medium with packaging cells 76c used for production of the selected virus 15. For example, in some embodiments, trigger event 350 or 360 is a time period associated with the cell culture medium such as the amount of time after inoculation of the cell culture medium with the packaging cells (i.e., the amount of time after an inoculum of packaging cells are added to the cell culture medium).

In other embodiments, trigger event 325 or 345 corresponds to a biological condition(s) of the cell culture medium and/or an amount or rate of change of the biological condition(s). In particular embodiments, trigger event 325 or 345 corresponds to a density of packaging cells within the cell culture medium. Typically, in such embodiments, the trigger event 350 is the reaching of a threshold upper limit of cell density and/or the reaching and maintaining of cell density above a lower limit and below an upper limit for a select period of time. In related embodiments, it may also correspond to an amount of change of cell density and/or a rate of change of cell density which, in either case, may be a minimum or maximum change or rate of change. Typically, the amount or rate of change will be a positive value reflecting a state of cell growth within the culture medium; however, in some embodiments, it may correspond to a negative value indicating, for example, that the cell culture had reached a plateau in growth and is now starting to decrease in cell density. The particular positive or negative value in the amount or rate of change of cell density used as a trigger event in a particular batch run can be selected based on one or more factors including the following: selected viral vector, packaging cell line, cell culture medium and volume of the bioreactor vessel. Cell density can be measured using various means known in the bioanalytical and bioprocessing arts, including, for example, optical means and electrical means. In particular embodiments, cell density is measured using optical turbidity or electrical capacitance techniques known in the art. In various embodiments, such turbidity, capacitance or other cell density measurements may be made continuously or at selected intervals under the control of a processor or other controller described herein.

In other embodiments, the biological condition used for the trigger event 350 or 360 may include one or more of pH, oxygen concentration, CO2 concentration or glucose concentration of the cell culture solution. Such measurements may also be selectively grouped together to create a composite or multifactor measurement of the cell culture medium providing information on an overall or multifactor state of cell culture where optimal transfection can occur.

In use, such embodiments using cell density and related properties as the trigger event allow the transfection solution including the transfection complexes to be added to the cell culture medium in a time window where optimal transfection efficiency of the packaging cells (with the plasmid/extrachromosomal DNA encoding the virus) can be achieved. (Transfection efficiency being defined in some embodiments as the number of packaging cells expressing the plasmid DNA encoding the virus divided by the total number of packaging cells in the cell culture medium.). In various embodiments, the triggering cell density can range from about one million to two million cells per ml with large and smaller ranges also considered depending on various factors. In various embodiments the triggering cell density can be selected based on one or more of the following factors: the cell line used for the packaging cells, the cell culture medium, the specific virus to be produced by the packaging cells, the desired viral concentration (e.g., one or both of physical and functional titer) or total amount of viral particles to be produced (e.g., 109 10 10 and the like), the size of the bioreactor vessel (2500 ml vs 5 L or 10 L or larger) or mixing speed within the bioreactor.

In many embodiments, the transfection solution 37 is delivered to the cell culture media within bioreactor vessel 75 at a set time period (e.g., minutes) after the trigger event where the time can be customized based on the particular virus to produced or other factor such as the packaging cell type, cell media batch, plasmids, transfection agent and size of the bioreactor vessel and/or desired amount of virus to be produced and/or desired viral production parameter. However, in alternative or additional embodiments, the delivery of the transfection solution 37 is initiated based on the second trigger event 360 which can be the same or different biological condition used for the first trigger event 350. When the biological conditions used for the two trigger events 350 and 360 are the same, there are two cases for the value of the biological condition used for the second trigger event. In the first case, the value for the biological condition for the second trigger event is the same or within a fixed percent range (+/−5%) of the value as the first trigger event. This case can be used where it is desirable not to have any significant change in the biological condition of the cell culture media after the first trigger event before the transfection solution is delivered. In the second case, the value for the biological condition for the second trigger point is above or below the value used for the first trigger point by set percentage (e.g., >5, 10, 20%) etc.). This case can be used where there is a change in the condition of the cells or cell media which may impact the transfection and/or viral production process, e.g., a drop in cell density.

In various embodiments, the packaging cell line used to generate the selected virus 15 corresponds to various mammalian cells known in the cell culture arts including one or more of embryonic cells, renal cells or embryonic renal cells one or more of which may be immortal cell lines. In particular embodiments, the mammalian cells used to generate the selected virus comprise HEK 293 cells and/or derivative cell lines known in the microbiological and cell culture arts. In still other embodiment, the cells used for the packaging cells may comprise other eukaryotic cells besides mammalian cells including yeast cells.

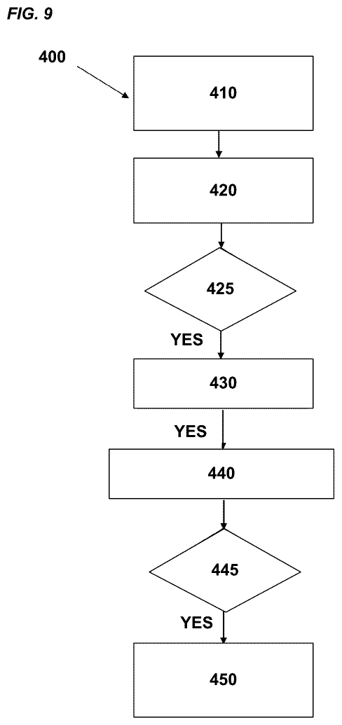

Referring now to FIG. 9, in addition to the embodiments of transfection methods described above, one specific embodiment of a method 400 of using an embodiment of transfection system 10 comprises the following. An operator fills the two syringes 22 and 42 with the reagents for the transfection process including plasmids and transfection reagent, typically PEI (or other lipid or polymer-based transfection reagents) in a filling step 410. These two reagents can then be diluted in cell culture media and dispensed into the syringes in a relatively time-insensitive manner, hours in advance of the transfection. In some embodiments, filling step 410 can be performed or aided by an automatic syringe filling system and/or a microfluidic chip. Then in step 420, the filled syringes are loaded into syringe pump 51 fluidically coupled to bioreactor vessel 75 or other component of system 10.

Once the syringes are loaded, the rest of the transfection process is controlled in an automated and pre-programmed way according to parameters chosen by the operator and/or a remote user. Upon an initial trigger event 425 which can be a specified time, or an output 780 from a specific sensor 78 (e.g., which senses cell density, metabolic activity, CO₂ levels etc.), in or operably coupled to the bioreactor, a mixing step 430 is initiated, in which one syringe plunger is depressed while the other is pulled out (which will typically be done by a dual action multi-syringe syringe pump). Combined with a pinch valve 81 blocking the path to the bioreactor vessel 75, this will cause the two reagents to mix together. The mixing rate and shear force experienced by the reagents are controlled by the pumping rate between the two syringes and thus are highly repeatable resulting in repeatable batches of transfection solution 37.

After mixing, in step 440 the combined reagents now making up transfection solution 37 are stored and/or incubated in one of the two syringes until the programmed transfection time. Periodic mixing in a user-defined schedule is also enabled by this system if desired, to the benefit of achieving the most effective transfection complexes possible.

Then upon a trigger event at 445 (e.g., cell density, time (in this case time after the mixing of solutions 30 and 35), or other sensed condition), the transfection solution 37 is delivered to the cell culture media 76 in bioreactor vessel 75 in a step 450 by opening the pinch valve and depressing the full syringe (either syringe 22 or 44). The pumping speed, shear forces, and timing are all automated (e.g., using a microprocessor or other programmable controller described herein) and thus repeatable, characterized, and consistent.

While various embodiments have been shown and described herein, it will be obvious to those skilled in the art that such embodiments are provided by way of example only. Numerous variations, changes, and substitutions will be apparent to those skilled in the art without departing from the disclosure. For example, embodiments of the transfection methods and systems described herein can be adapted for multiple cell types, including mammalian, bacterial and yeast cells. They can also be adapted for use on a variety of scales and settings including, for example, a research laboratory, GMP production facility and hospital or other point of care setting. Further, they may be adapted for both batch and perfusion-based bioreactors and cell culture methods. They may also be adapted for production of viral vectors for a number of different clinical applications and research purposes including one or more of gene therapy, oncolytic virus production, vaccine production or biopharmaceutical production. It should be understood that various alternatives to the embodiments described herein may be employed for transfection of cells with cellular or other non-viral DNA, for example, to reprogram the target cells to generate a selected protein or express a desired cell surface antigen or receptor. Alternative embodiments may also be adapted for transfection of yeast or other non-mammalian eukaryotic cells for production of viral vectors.

Elements, characteristics, or acts from one embodiment can be readily recombined or substituted with one or more elements, characteristics or acts from other embodiments to form numerous additional embodiments within the scope. Moreover, elements that are shown or described as being combined with other elements (e.g., plasmids, cells, viruses, virions, chemical, mechanical component, and the like.), characteristics, steps or acts can, in various embodiments, exist as stand-alone elements, characteristics, steps or acts. Further, various embodiments expressly contemplate the negative recitation of any element, characteristic, step or act etc. that is/are shown or described in one or more embodiments. Accordingly based on the above considerations, the scope is not limited to the specifics of the described embodiments but is instead limited solely by the appended claims.

The terms “substantially” and “about” are used herein to describe and account for small variations including small variations in a recited, parameter, property, quality, or dimension. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely intended to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments, and does not pose a limitation to the scope of the disclosure unless otherwise claimed. No language in the specification should be construed as indicating any non-claimed element as essential to any embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

What is claimed is:

1. A method for transfecting cells to produce a selected virus, the method comprising:
providing a first solution comprising extrachromosomal DNA molecules encoding the virus;
providing a second solution comprising a transfection agent; wherein the first and second solutions are kept separate;
mixing the first and second solutions to produce a transfection solution wherein the mixing is initiated responsive to a trigger event; the transfection solution to be delivered to a cell culture medium for transfection of cells in the medium with the extrachromosomal DNA molecules encoding the virus;
incubating the transfection solution to form transfection complexes configured to transfect the cells in the medium; and
delivering the transfection solution to the cell culture medium to transfect the cells in the medium wherein a viral production parameter in a viral production batch resulting from transfection of the cells is optimized by initiating the mixing of the first and second solutions responsive to the trigger event.

2. The method of claim 1, wherein the viral production parameter is at least one of a viral concentration, a batch-to-batch variability of viral concentration, an amount of variation in the genetic composition of a population of viral particles from a production batch, an average virus infectivity of a viral production batch.

3. The method of claim 2, where the viral concentration comprises at least one of a physical titer or a functional titer of virus.

4. The method of claim 1, wherein the selected virus comprises a genetically modified virus, an adenovirus, an adeno-associated virus, a baculovirus, a herpes simplex-1 virus, a lentivirus or a retrovirus.

5. The method of claim 1, wherein the extrachromosomal DNA molecules comprises plasmids.

6. The method of claim 1, wherein the transfection agent comprises at least one of a lipid, a polymer or lipofectamine.

7. The method of claim 1, wherein the transfection complexes comprise a plurality liposome complexes comprising liposomes entrapping the extrachromosomal DNA molecules.

8. The method of claim 1, wherein the trigger event is a time period associated with the cell culture medium.

9. The method of claim 8, wherein the time period is an amount of time after rate of inoculation of the cell culture medium with the cells.

10. The method of claim 1, wherein the trigger event is a biological condition of the cell culture medium.

11. The method of claim 10, wherein the trigger even is an amount of change of the biological condition.

12. The method of claim 10, wherein the trigger even is rate of change of the biological condition.

13. The method of claim 10, wherein the biological condition is a cell density of the cell culture medium.

14. The method of claim 10, wherein the cell density is a viable cell density.

15. The method of claim 14, wherein the cell density is in a range from about one million to four million cells per ml.

16. The method of claim 15, wherein the cell density is in a range from about two million to four million cells per ml.

17. The method of claim 14, wherein the cell culture medium is perfused during at least a portion of a growth period of cells within the medium, and wherein the cell density is in a range from about one million to ten million cells per ml.

18. The method of claim 17, wherein the cell density is about four million cells per ml.

19. The method of claim 17, wherein the cell density is in a range from about six million to ten million cells per ml.

20. The method of claim 13, wherein the cell density is measured continuously.

21. The method of claim 13, wherein the cell density is measured optically.

22. The method of claim 21, wherein the cell density is measured using a turbidity measurement of the cell culture medium.

23. The method of claim 14, wherein the cell density is measured electrically.

24. The method of claim 23, wherein the cell density is measured using a capacitance measurement of the cell culture medium.

25. The method of claim 10, wherein the biological condition is at least one of a pH, an oxygen concentration, a glucose concentration.

26. The method of claim 1, wherein the cells comprise mammalian cells.

27. The method of claim 26, wherein the mammalian cells comprise embryonic cells, renal cells or embryonic renal cells.

28. The method of claim 26, wherein the mammalian cells comprise HEK 293 cells.

29. The method of claim 1, wherein the first and second solutions are mixed for a selected time period.

30. The method of claim 29, wherein the period is between about 0.5 to 5 minutes.

31. The method of claim 30, wherein the period is between about 0.5 to 1 minutes.

32. The method of claim 1, wherein the transfection solution is incubated for a selected time period.

33. The method of claim 24, wherein the time period is between about 1 to 60 minutes.

34. The method of claim 33, wherein the time period is between about 5 to 20 minutes.

35. The method of claim 1, wherein the transfection solution is delivered to the cell culture medium at a selected time period after the trigger event.

36. The method of claim 35, wherein the period is between about 0.1 to 5 minutes.

37. The method of claim 1, wherein the selected time period after the trigger event is below a period at which the transfection complexes undergoes degradation.

38. The method of claim 1, wherein the first and second solutions are contained in first and second chambers.

39. The method of claim 1, wherein the chambers comprise syringes.

40. The method of claim 38, wherein mixing comprises cyclically flowing the first and second solutions back and forth between the first and second chambers.

41. The method of claim 38, wherein the cyclical flow is controlled by a controller including a set of electronic instructions for controlling the flow.

42. The method of claim 39, wherein the first and second solutions are mixed for a selectable number of cycles.

43. The method of claim 42, wherein the selectable number of cycles is between about 3 to 4.

44. The method of claim 39, wherein a flow rate between the first and second chambers is configured to maintain a fluid shear stress or strain rate of the flow of the solutions between chambers below a selected level.

45. The method of claim 44, wherein the transfection agent comprises plasmids and the flow rate between the first and second chambers is configured to maintain a fluid shear stress or strain rate of the flow of the solutions between chambers below a level causing rupture or damage of the plasmids.

46. The method of claim 45, wherein the shear stress is below about 40 dynes/cm².

47. The method of claim 46, wherein the shear stress is below about 16 dynes/cm².

48. The method of claim 45, wherein the strain rate is below about 1.5×104 sec⁻¹.

49. The method of claim 48, wherein the strain rate is below about 7×103 sec⁻¹.

50. The method of claim 1, wherein one or more steps of the method is controlled by a controller comprising a set of electronic instructions for controlling the one or more steps.

51. The method of claim 50, wherein the controller comprises a processor.

52. The method of claim 50, wherein the electronic instruction set comprises a software module.

53. The method of claim 1, wherein one or more steps of the method are performed or controlled using an automated filling device.

54. The method of claim 53, wherein the one or more steps comprise at least one of mixing of the first and second solutions and delivery of the transfection solution.

55. The method of claim 1, wherein one or more steps of the method are performed on a microfluidic chip.

56. The method of claim 55, wherein the one or more steps comprise at least one of mixing of the first and second solutions, incubation of the first and second solutions and delivery of the transfection solution.

57. The method of claim 1, the cell culture medium is at least partially contained in a bioreactor vessel.

58. The method of claim 1, further comprising providing at least a third solution.

59. The method of claim 58, wherein the least a third solution comprises extrachromosomal DNA molecules or transfection reagent.

60. The method of claim 59, wherein the extrachromosomal DNA molecules in the third solution are different from that in the first solution.

61. The method of claim 59, wherein the transfection reagent in the third is different from that in the second solution.

62. A system for transfecting cells to produce a selected virus, the system comprising:
a first fluid delivery device configured to contain and deliver a first solution comprising extrachromosomal DNA molecules encoding the virus;
a second fluid delivery device configured to contain and deliver a second solution comprising a transfection agent;
a first fluid delivery channel fluidically coupled to the first and second fluid delivery devices;
at control valve fluidically coupled to at least one of the first and second fluid delivery devices and the first fluidic delivery channel, the control valve configured to control flow between the first and second fluid delivery devices and outward flow from one or both of the fluid deliver devices to another fluid delivery channel;
a second fluid delivery channel fluidically coupled to the at least one control valve, the second fluid delivery channel configured to be fluidically coupled to a bioreactor vessel, the bioreactor vessel configured to contain the cells to be transfected; and
a controller operatively to the control valve, the controller configured to control flow between the first and second chambers and flow from at least one of the first and second chambers to the second fluid delivery channel.

63. The system of claim 62, further comprising a drive means coupled to at least one of the first and second fluid delivery devices, the drive means configured to apply a driving force to the first and second fluid delivery devices.

64. The system of claim 62, where in the first and second delivery devices comprise first and second syringes.

65. The system of claim 64, further comprising a syringe drive means coupled to the first and second syringes.

66. The system of claim 62, where the controller comprises a processor.

67. The system of claim 66, wherein the processor includes a software module for controlling flow between the flow between the first and second chambers and flow from at least one of the first and second chambers to at least one of the second fluid delivery channel or the bioreactor vessel.

68. The system of claim 62, wherein the control valve comprises a three-way valve.

69. The system of claim 62, further comprising a mixing device fluidically coupled to at least one of the first and second delivery devices and the first fluidic channel, the mixing device configured to mix the first and second solutions upon flow of the solutions by or through the mixing device.

70. The system of claim 62, further comprising the bioreactor vessel.

71. The system of claim 70, at least one sensor operatively coupled to the bioreactor vessel and the controller.

72. The system of claim 71, wherein the at least one sensor is disposed within the bioreactor.

73. The system of claim 71, wherein the at least one sensor is configured to measure a cell density within the bioreactor vessel.

74. The system of claim 73, wherein the at least one sensor is an optical sensor.

75. The system of claim 71, wherein optical sensor is configured to measure turbidity of a solution within the bioreactor vessel.

76. The system of claim 73, wherein the at least one sensor is an electrical sensor.

77. The system of claim 76, wherein the electrical sensor is configured to measure a capacitance of a solution within the bioreactor.

78. The system of claim 62, wherein one or more components of the system are disposed in or on a micro-fluidic chip.
